(19) United States
(12) Patent Application Publication
(10) Pub. No.: US 2026/0130780 A1
(43) Pub. Date: May 14, 2026

(54) SENSOR DEVICE

(71) Applicant: Otto Bock Healthcare Products GmbH, Vienna (AT)

(72) Inventors: Danijel Djordjevic, Vienna (AT); Andreas Bohland, Vienna (AT); Malte Asseln, Vienna (AT)

(21) Appl. No.: 19/119,319

(22) PCT Filed: Oct. 10, 2022
(86) PCT No.: PCT/EP2022/078152

§ 371 Date: Apr. 8, 2025

(51) Int. Cl. A61F 5/01; A61F 2/02; A61F 2/50

(52) U.S. Cl. CPC A61F 5/01; A61F 2/02; A61F 2/50; A61F 2002/5081

(57) ABSTRACT

The invention relates to a sensor device comprising at least two sensors (12) and a main body (18) with an upper part (2) and a lower part (4) and at least one elastic element (6) arranged between the upper part (2) and the lower part (4), wherein the upper part (2) can be displaced relative to the lower part (4) from a zero position along a preferred direction and tilted about a tilt axis perpendicular to the preferred direction, wherein at least one elastic element (6) is deformed and the at least two sensors (12) are each configured to determine a distance between the upper part (2) and the lower part (4), so that a displacement and/or tilt of the upper part (2) relative to the lower part (4) from the zero position can be determined.

The invention relates to a sensor device comprising at least two sensors and a main body with an upper part and a lower part and at least one elastic element arranged between the upper part and the lower part.

Such sensor devices are known from the prior art. For example, WO 2020/041491 A1 describes such an arrangement that is arranged in an orthopedic device, for example in a connecting element for connecting two components of a prosthesis. The device has an upper part on which a conventional pyramid adapter is located for connecting it to another prosthesis component. The lower part is designed as a plate-shaped element and is connected to the upper part via a centrally arranged bar. This allows the upper part to be tilted relative to the lower part when the central bar is deformed. Sensors are arranged on the respective outer edges of the upper and lower parts which can measure a distance between the upper part and the lower part. This allows an angle between the upper part and the lower part to be determined. Said angle occurs when different forces prevail on opposite sides of the bar. The disadvantage is that very precise sensors are required due to the low deflection and the end stops of the deflection are reached early.

A similar device is known from WO 2021/040998A1. It has a frame that can be deformed when forces act on it. This device is also intended for use in orthopedic devices. It has a base which bears the pyramid adapter for connecting to other prosthesis components and forms the lower part. This base has an elevation extending from medial to lateral, on which the frame that forms the upper part rests. Anterior and posterior to this elevation there is a small gap between the upper part and the lower part, which allows the upper part to move under the effect of forces, wherein this movement causes a deformation of the upper part. In this embodiment, it is only possible to determine forces that cause a deformation of the upper part.

U.S. Pat. No. 8,746,080 B2 discloses a connecting device for orthopedic devices that comprises a pyramid adapter with a cavity. Under high loads, this results in deformations of the adapter and thus the cavity which can be determined by distance sensors. In this case too, the forces have to cause a deformation of the upper part, which is formed by the upper part of the adapter, in order for a measurement to take place.

The invention is therefore based on the task of eliminating or at least reducing the disadvantages of the prior art.

The invention solves the task addressed by way of a sensor device comprising at least two sensors and a main body with an upper part and a lower part and at least one elastic element arranged between the upper part and the lower part, wherein the sensor device is characterized in that the upper part can be displaced relative to the lower part from a zero position along a preferred direction and tilted about a tilt axis perpendicular to the preferred direction, wherein at least one elastic element is deformed and the at least two sensors are each configured to determine a distance between the upper part and the lower part, so that a displacement and/or tilt of the upper part relative to the lower part from the zero position can be determined.

Unlike in embodiments known from the prior art, with the embodiment according to the invention, the upper part can also be displaced relative to the lower part along the preferred direction, preferably without this leading to a deformation of the upper part. The at least two sensors are configured to determine a distance between the upper part and lower part, wherein this happens at two different locations. In advantageous embodiments, there are more than two, preferably more than three, particularly preferably more than four sensors, each of which determines a distance between the upper part and the lower part. The positions at which this occurs are preferably arranged such that they are not in a straight line.

The sensors do not have to be able to quantitatively determine a distance between the upper part and the lower part, even if this does constitute a preferred embodiment. For the function of the invention, it is sufficient for each sensor to be able to determine whether there is a distance between the upper part and the lower part at its position or not. Such a sensor can therefore be designed as a contact sensor or pressure sensor, for example, or it can be a different type of sensor that can recognize whether the upper part is resting on the lower part.

d

Preferably, the size of the distances between the upper part and the lower part is known for each individual sensor in the unloaded state. This corresponds to a zero position. The individual distances need not be the same for all sensors in the unloaded state. However, it is advantageous if they are. If a force now acts on the upper part and/or the lower part that leads to a movement of the upper part relative to the lower part, at least some, but usually all, distances between the upper and lower part that each are measured by one sensor change. If all distances change equally, this means that the upper part and the lower part have been moved towards each other without the orientation of the upper part relative to the lower part changing. Consequently, this constitutes a displacement along the preferred direction.

However, if the distances between the upper part and the lower part change in different ways at different positions, the orientation of the upper part to the lower part also changes. Consequently, this constitutes a tilt about the tilt axis. Of course, the two movements can also be superimposed, for example if all the distances between the upper and the lower part detected by the sensors become smaller but are reduced by different amounts.

In the embodiment of the sensor device according to the invention, unlike in the prior art, it is not the upper part or the lower part that is deformed, but an elastic element preferably arranged between them. Preferably, there is more than one elastic element, for example two, three or four elastic elements, which are deformed upon a movement of the upper part relative to the lower part. In this case, it is not necessary for all elastic elements to be deformed with each movement. The elastic elements are arranged in such a way that the strength and type of deformation which has taken place in the elastic elements can be determined from the measurement result of the sensors that determine the distance between upper and lower part. The spring constants, i.e. the relationship between the deformation of an elastic element and the force that causes it, are preferably known for all elastic elements. This relationship is also referred to as a spring constant when the relationship is not a linear one. The magnitude and direction of the force acting on the sensor device can then be determined from the knowledge of all these variables.

The use of at least one elastic element allows greater displacements and/or tilts of the upper part relative to the lower part over the force and/or torque range to be measured than is the case in embodiments from the prior art. As a result, sensors with a lower resolution and measurement accuracy can also be used, which are usually cheaper. Sufficient mechanical stability of the sensor device is preferably ensured by end stops. When reached, large forces and/or torques can be transferred from the upper part to the lower part without the at least one elastic element being exposed to excessive loads.

In one preferred embodiment, the at least one elastic element opposes a tilt of the upper part relative to the lower part in a first tilt direction with a different resistance than it opposes a tilt in a second tilt direction. This is advantageous, for example, when the anticipated forces and/or resulting and generated torques are different sizes in different tilt directions.

Preferably, the upper part can be displaced relative to the lower part along a plane of symmetry of the main body, preferably a plane of symmetry of the sensor device. The sensor device is preferably arranged in an orthopedic device, such as an orthosis or a prosthesis, in order to determine the forces and torques that occur there. The main body plane of symmetry, preferably the sensor device plane of symmetry, is then preferably arranged in such a way that it corresponds to a frontal plane or a sagittal plane of the wearer of the orthopedic device. The preferred direction, along which the upper part can be displaced relative to the lower part and which lies in the plane of symmetry, then preferably extends from proximal to distal.

The tilt axis preferably lies in the plane of symmetry. The plane of symmetry is then preferably spanned by the tilt axis and the preferred direction.

Advantageously, the at least two sensors are on opposite sides of the plane of symmetry and are preferably at the same distance from said plane of symmetry. This renders the calculation of the acting forces from the distance measurements of the sensors especially easy. In the event that the tilt axis is not in the plane of symmetry, but, for example, is tilted in relation to it, it is advantageous for the sensors to be at the same distance from the tilt axis.

Advantageously, the upper part is connected to the lower part by at least one connecting bar. The at least one elastic element then comprises hinges, for example film hinges, by means of which the connecting bar is connected to the upper part and the lower part. If the upper part now moves relative to the lower part, the positions of the hinges change. In order to keep the connecting bars connected to the upper part and the lower part, the hinge also has to move and assume a different angle. The hinge, especially in the form of a film hinge, sets an elastic force against this movement of the hinge. Consequently, the movement of the hinge corresponds to the deformation of the elastic element. Depending on the configuration of the hinges, especially the film hinges, the force required to deform the elastic element can be of varying size.

In one preferred embodiment, there is at least one connecting bar on each side of the plane of symmetry and/or the tilt axis. Each of the hinges has two elements that can be twisted or swivelled relative to each other, said elements being referred to as tabs or lugs. In each case, one of these tabs of each hinge is connected to the connecting bar, while the other of the two tabs is connected to the upper part or lower part. If the hinge is a film hinge, the two tabs are preferably designed as a single piece. In one especially preferred embodiment, the upper part, lower part, connecting bars and hinges are designed as a single piece.

The sensor device preferably has at least two end stops, each of which limit a tilt of the upper part relative to the lower part in one direction. This prevents overstressing and overloading of the elastic elements. However, this means that torques which lead to a tilt of the upper part relative to the lower part in the respective direction can only be measured or determined up to a maximum value.

Preferably, the end stops are arranged on the lower part and the upper part comes into contact with these end stops as soon as it has been swivelled relative to the lower part far enough in the respective direction. To this end, a contact element, for example in the form of a hardened plate, can be arranged on the upper part. Of course, the reverse arrangement can also be used, i.e. with the end stop on the upper part and the contact element on the lower part. In one particularly preferred embodiment, one of the at least two sensors is located in the contact element or in one of the end stops.

Preferably, the sensor device has at least one additional end stop that limits the displacement of the upper part relative to the lower part. The additional end stop is preferably between the two end stops. Particularly preferably, the additional end stop is located in the preferred direction. It is highly preferable if the additional end stop is located in the plane of symmetry of the main body, preferably in the plane of symmetry of the sensor device. In principle, the displacement of the upper part relative to the lower part is also limited by the two end stops, which also limit tilting. If one of the end stops is resting on the respective contact element, further tilting in the respective direction is not possible. If both end stops are resting on their respective contact element, a further displacement of the upper part relative to the lower part is not possible. However, it is then not possible to determine whether, in addition to the large force, a possibly smaller torque is also acting on the upper part or the sensor device. This is only achieved with an additional end stop. However, the additional end stop is preferably designed in such a way that, when the contact element of the additional end stop is resting on it, further tilting of the upper part relative to the lower is possible.

Particularly preferably, the additional end stop has a rolling contour so that the upper part can roll on the lower part or vice versa. This facilitates further tilting, even if a further displacement has already been ruled out by the additional end stop. Of course, the additional end stop may also be arranged on the upper part or the lower part and the respective contact element on the respective other component of the main body.

In one preferred embodiment, the sensor device has an additional sensor that is arranged in the plane of symmetry and configured to determine the distance between the upper part and the lower part. This additional sensor is preferably in the additional end stop or its contact element.

Advantageously, the at least two sensors and/or the additional sensor include at least one Hall sensor and/or at least one optical sensor. With the at least two sensors and/or the additional sensor, it is possible to measure the distance contactlessly.

In one preferred embodiment, a permanent magnet is arranged on the upper part or lower part of the main body. At least two Hall sensors are positioned on the respective other part in such a way that they are in the magnetic field of the permanent magnet. If a force is now exerted on the upper part of the main body, it results in a displacement and/or tilt of the upper part, which results in a movement of the permanent magnet relative to the two Hall sensors. The two sensors are preferably arranged in such a way that a pure displacement of the upper part relative to the lower part causes the distance between the permanent magnet and the two Hall sensors to change to the same extent. The sensors then detect a change in the magnetic field, wherein this change is likewise the same for both Hall sensors. As a result, it is possible to detect that it is only a displacement of the upper part relative to the lower part, i.e. a longitudinal movement. If the force acting on the upper part and/or the lower part also leads to a tilt of the upper part relative to the lower part, it results in the distance between the permanent magnet and the two Hall sensors not changing to the same, but a different degree for both sensors. This also changes the magnetic field detected by the two sensors to different degrees. The tilt angle about a particular axis can thus be determined.

This configuration of the at least two sensors has the advantage that it directly measures a tilt and/or displacement of the upper part relative to the lower part, irrespective of the structure of the main body. Here, an elastic element is advantageous, but not essential. A sensor device comprising at least two Hall sensors and a main body with an upper part and a lower part, the Hall sensors being arranged on the upper part and a permanent magnet being arranged on the lower part or the Hall sensors being arranged on the lower part and the permanent magnet on the upper part, and the sensor device being characterized in that the upper part can be displaced relative to the lower part from a zero position along a preferred direction and tilted about a tilt axis perpendicular to the preferred direction, wherein the at least two Hall sensors are each configured to determine a distance between the upper part and the lower part, so that a displacement and/or tilt of the upper part relative to the lower part from the zero position can be determined, thus represents a separate invention. This separate invention can be combined with all other features that are described in the present case and do not refer to the at least one elastic element.

Particularly preferably, the at least two Hall sensors are positioned symmetrically to the plane of symmetry of the main body, preferably symmetrically to the plane of symmetry of the entire sensor device. In one particularly preferred embodiment, there are at least three, especially preferably at least four Hall sensors, which are not positioned along a single straight line. In this way, a tilt about each axis can be detected, which results in different degrees of change in the magnetic fields detected by the Hall sensors used.

Preferably, the at least two sensors and/or the additional sensor include at least one contact sensor, a pressure sensor and/or a capacitive sensor. Such a sensor is preferably arranged and configured in such a way that it is able to determine whether there is a distance between the upper part and lower part or whether the upper part and lower part are touching.

In the following, a number of embodiment examples of the invention will be explained in more detail with the aid of the accompanying figures. They show

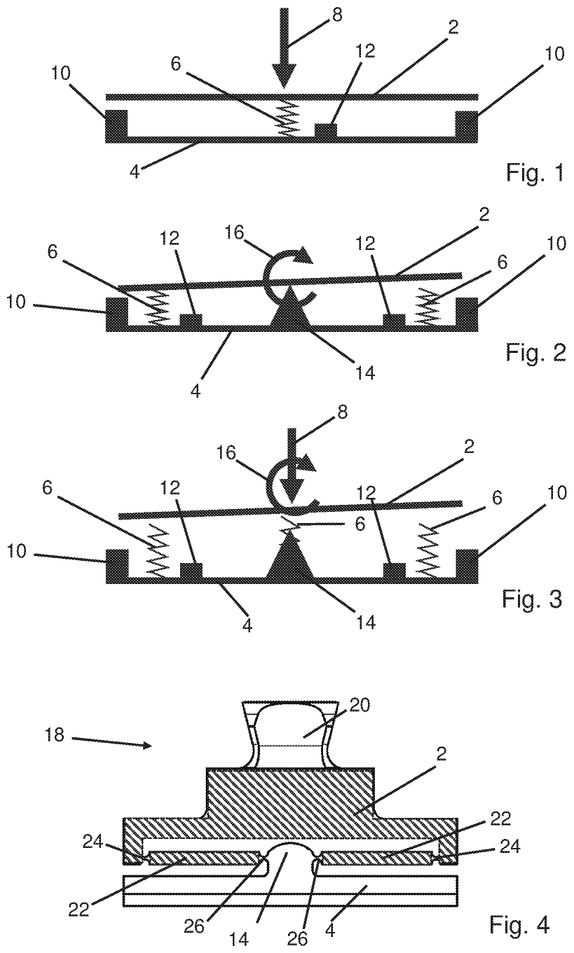

FIGS. 1 to 3 schematic representations of modes of operation that are realized in the present invention,

FIGS. 4 to 7 schematic representations of a main body under the influence of different forces and

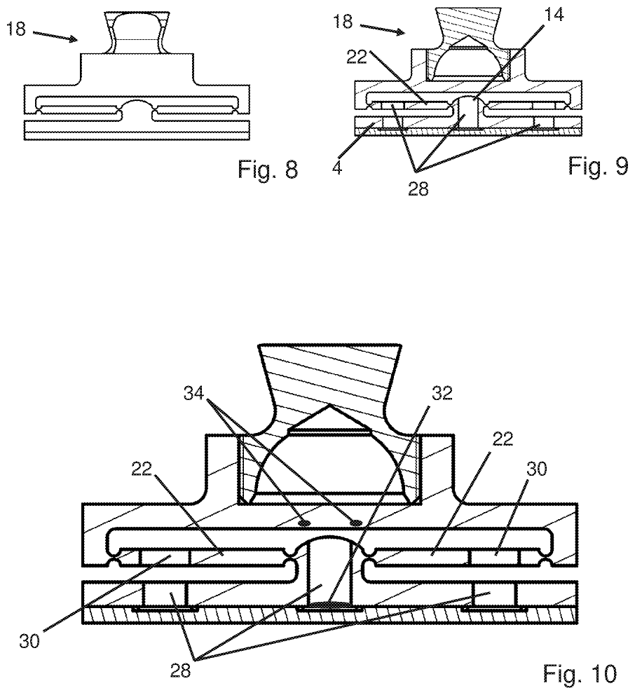

FIGS. 8 to 10 the schematic side view, sectional representation and sensor positioning of a main body.

FIG. 1 schematically depicts a function realized in a sensor device according to an embodiment example of the present invention. An upper part 2 and a lower part 4 of a main body are illustrated. There is an elastic element 6 between the two, which is designed as a spring in the schematic representation shown. If a force 8, represented by the arrow, now acts in the direction opposite the arrow, the elastic element 6 is compressed. The configuration and direction of the force 8 causes the upper part 2 to move relative to the lower part 4 along the direction of the acting force 8. FIG. 1 illustrates the situation in which only a displacement occurs between the upper part 2 and the lower part 4.

In this situation, there are two end stops 10 by means of which the maximum possible displacement is limited. This is the case when the upper part 2 strikes the end stops 10. A sensor 12 is shown between the upper part 2 and the lower part 4 by means of which the distance between the upper part 2 and the lower part 2 can be measured.

FIG. 2 illustrates a situation in which only a tilt of the upper part 2 relative to the lower part 4 is possible. Instead of the one elastic element 6, which is arranged in the center in FIG. 1, two elastic elements 6 are depicted in FIG. 2. There is an additional end stop 14 in the center on which the upper part rests. This representation was selected in order to allow only a tilt, but not a displacement, of the upper part 4 relative to the lower part 4. The end stops 10 are again provided on both sides. Unlike in FIG. 1, in which the upper part 2 could only come into contact with both end stops 10 by displacing it, in FIG. 2 the upper part 2 can only come into contact with one of the two end stops 10. This is possible when a torque 16 is acting, which is represented by the curved arrow. The arrow only serves to show that a torque is acting. The direction of this arrow is not supposed to determine the direction of the torque.

The arrangement shown in FIG. 2 has two sensors 12, each of which is configured to determine the distance between the upper part 2 and the lower part 4 at its position. The angle of the upper part 2 relative to the lower part 4 can be determined using the different distances between the upper part 2 and the lower part 4 and the position of the sensors 12, and thus the effective torque can be determined from the known force curves of the elastic elements 6. In FIG. 2, it is already visible that an elastic element 6 can also disengage from the upper part 2 if, for example, this is brought on by a swivelling caused by a torque 16. In FIG. 2, this is the case with the elastic element 6 on the right.

FIG. 3 shows the combination of the two situations from FIG. 1 and FIG. 2. This corresponds functionally to a sensor device according to an embodiment of the present invention. In this case, three elastic elements 6 are positioned between the upper part 2 and the lower part 4. They are compressed to different degrees when a force 8 and/or a torque 16 is acting. In the embodiment example shown, the two lateral elastic elements 6 are not yet in contact with the upper part 2. However, this is not necessarily the case. Embodiments in which the lateral elastic elements 6 are in contact with the upper part 2 can be equally advantageous in certain situations.

The embodiment depicted in FIG. 3 features the two end stops 10, the two sensors 12 and the additional end stop 14.

FIG. 4 shows an embodiment of a main body 18 for a sensor device according to an embodiment example of the present invention. The main body has a pyramid adapter 20, which forms part of the upper part 2. The lower part 4 comprises the additional end stop 14. In FIG. 4, there are two connecting bars 22 between the upper part 2 and the additional end stop 14 of the lower part 4, wherein the outer end of said bars in FIG. 4 is connected to the upper part 2 via a film hinge 24. The inner end of the connecting bars 22 in FIG. 4 is connected to the additional end stop 14 of the lower part 4 via a further film hinge 26.

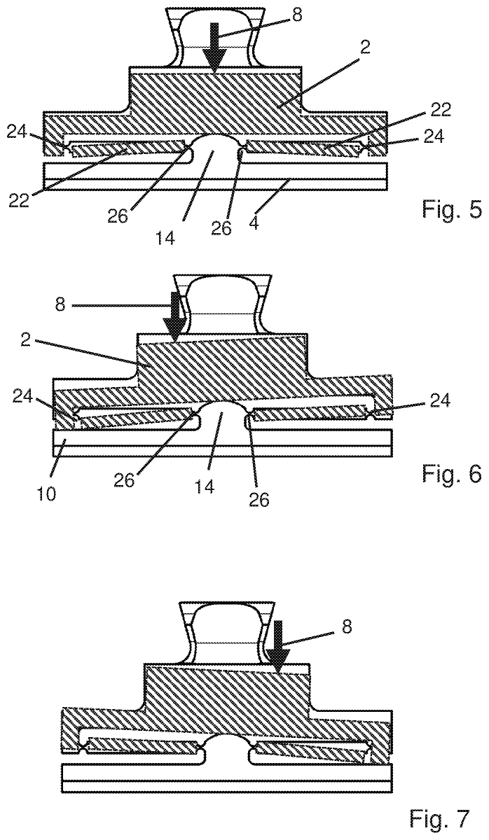

FIG. 5 shows the configuration from FIG. 4 under the influence of a force 8. One can see that the upper part 2 has been displaced downwards relative to the lower part 4, so that the connecting bars 22 no longer extend parallel to each other or to the lower part 4. To reach this position, the film hinges 24, 26 have been elastically deformed. In this case, they form the elastic elements 6. The displacement of the upper part 2 relative to the lower part 4, which was caused by the force 8, ended when the upper part 2 was resting on the additional end stop 14.

FIG. 6 depicts the situation from FIG. 5, wherein the force 8 is no longer central, unlike in FIG. 5, i.e. it no longer acts along the axis of symmetry of the main body 18, but at an offset to it. This also results in a torque 16 which, however, is not graphically depicted. The upper part 2 has been both displaced and swivelled from the rest position shown in FIG. 4 by the force acting on it. The upper part is resting on the additional end stop 14 and the end stop 10 on the left. In this example too, the film hinges 24, 26 have been deformed and form the elastic elements. The respective reverse situation is depicted in FIG. 7 in which the force 8 is not acting to the left of the axis of symmetry as in FIG. 6, but to the right of the axis of symmetry of the main body 18. FIGS. 5 and 6 in particular show that the upper contact surface of the additional end stop 14 is curved and designed as a rolling surface, so that the upper part 2 can be tilted particularly easily even when it is already in contact with the additional end stop 14.

FIG. 8 depicts a side view of a main body 18 as it has already been depicted in FIG. 4. FIG. 9, however, shows a sectional representation through the main body 18 from FIG. 8. Measuring channels 28 that begin in the lower part 4 are visible. A central measuring channel 28 runs through the additional end stop 14 and is designed to be open at the top in the direction of the upper part 2. The two measuring channels 28 at the side also begin in the lower part 4 and extend through openings in the connecting bar 22.

FIG. 10 shows the enlarged representation from FIG. 9. The measuring channels 28 and the openings 30 in the connecting bar 22 are visible. A permanent magnet 32 is located at the bottom of the center measuring channel 28, the magnetic field of which extends in particular through the center measuring channel 28. Two Hall sensors 34 are arranged on the side of the upper part 2 that faces the lower part 4. They measure the magnetic field of the permanent magnet 32 very precisely, enabling them to detect changes in the distance between the upper part 2 and the lower part 4 very precisely. Due to their arrangement, they can also detect if the change at the two respective positions of the Hall sensors 34 is different. This allows a tilt to be calculated. Additionally or alternatively, sensors may also be arranged in the lateral measuring channels 28.

### Reference List

2 upper part
4 lower part
6 elastic element
8 force
10 end stop
12 sensor
14 additional end stop
16 torque
18 main body
20 pyramid adapter
22 connecting bar
24 film hinge
26 film hinge
28 measuring channel
30 opening
32 permanent magnet
34 Hall sensor

1. A sensor device comprising
at least two sensors (12) and
a main body (18) with an upper part (2) and a lower part (4) and at least one elastic element (6) arranged between the upper part (2) and the lower part (4),
characterized in that
the upper part (2) can be displaced relative to the lower part (4) from a zero position along a preferred direction and tilted about a tilt axis perpendicular to the preferred direction, wherein at least one elastic element (6) is deformed and the at least two sensors (12) are each configured to determine a distance between the upper part (2) and the lower part (4), so that a displacement and/or tilt of the upper part (2) relative to the lower part (4) from the zero position can be determined.

2. The sensor device according to claim 1, characterized in that the at least one elastic element (6) opposes a tilt of the upper part (2) relative to the lower part (4) in a first tilt direction with a different resistance to a tilt in a second tilt direction.

3. The sensor device according to claim 1, characterized in that the upper part (2) can be displaced relative to the lower part (4) along a plane of symmetry of the main body (18), preferably a plane of symmetry of the sensor device.

4. The sensor device according to claim 3, characterized in that the tilt axis lies in the plane of symmetry.

5. The sensor device according to claim 3, characterized in that the two sensors (12) are on opposite sides of the plane of symmetry and are preferably at the same distance from said plane of symmetry.

6. The sensor device according to claim 1, characterized in that the upper part (2) is connected to the lower part (4) by at least one connecting bar (22) and the at least one elastic element (6) comprises hinges, especially film hinges (24,26), by means of which the connecting bar (22) is connected to the upper part (2) and the lower part (4).

7. The sensor device according to claim 1, characterized in that the sensor arrangement has at least two end stops (10) each of which limits a tilt of the upper part (2) relative to the lower part (4) in one direction.

8. The sensor device according to claim 1, characterized in that the sensor arrangement has at least one additional end stop (14) by means of which the displacement of the upper part (2) relative to the lower part (4) is limited.

9. The sensor device according to claim 8, characterized in that the additional end stop (14) has a rolling contour on which the upper part (2) can roll on the lower part (4).

10. The sensor device according to claim 3, characterized in that the sensor device has an additional sensor that is arranged in the plane of symmetry and configured to determine the distance between the upper part (2) and the lower part (4).

11. The sensor device according to one claim 1, characterized in that the at least two sensors (12) and/or the additional sensor include at least one Hall sensor (34) and/or at least one optical sensor.

12. The sensor device according to claim 1, characterized in that the at least two sensors (12) and/or the additional sensor include at least one contact sensor, a pressure sensor and/or at least one capacitive sensor.
